(19) United States
(12) Patent Application Publication
(10) Pub. No.: US 2024/0286104 A1
(43) Pub. Date: Aug. 29, 2024

(54) POLYANILINE COATED ELECTRODES FOR POLYMER SYNTHESIS

(71) Applicant: Lenovo (Singapore} Pte, Ltd., Singapore (SG)

(72) Inventors: Marilia Santos Menossi Mortari, Butanta (BR); Sergio Matias Pereira Junior, Butanta (BR); Caio Perez Gomes, Butanta (BR); Ariane Simões Tunussi, Butanta (BR); Bruno Ramos, Butanta (BR)

(21) Appl. No.: 18/174,105

(22) Filed: Feb. 24, 2023

(51) Int. Cl. B01J 19/00; C07K 1/04

(52) U.S. Cl. CPC B01J 19/0046; C07K 1/045; B01J 2219/00612; B01J 2219/00659; B01J 2219/00722

(57) ABSTRACT

Various aspects disclosed relate to forming a coating on an electrode of an oligonucleotide syntheses apparatus. The coating can include a polyaniline-containing material. The coating can be formed by applying a voltage to the electrode under specified conditions in the presence of a solution including an amount of acid content and an amount of aniline monomer units.

### BACKGROUND

Polymers can be synthesized by the addition of monomer units to form macromolecules having a number of repeating subunits. Polymers can be formed through synthetic processes and through natural processes. In one or more examples, biopolymers can be formed within an organism through a number of biochemical reactions. In some cases, biopolymers can be synthesized outside of an organism via at least one of one or more chemical processes or one or more electrochemical processes.

### SUMMARY OF THE DISCLOSURE

One or more aspects disclosed relate to an oligonucleotide synthesis apparatus. The oligonucleotide synthesis apparatus includes a reaction vessel. The oligonucleotide synthesis apparatus also includes an array of electrodes disposed in the reaction vessel. Individual electrodes of the array of electrodes have a coating that includes a polyaniline-containing material.

In addition, one or more aspects disclosed relate to a method of forming a coating on an electrode used to synthesize oligonucleotides. The method includes providing a substrate with one or more electrodes disposed on the substrate. The method also includes contacting the one or more electrodes with a coating solution. The coating solution has an acid content and a monomer content. The monomer content of the coating solution includes a number of aniline monomer units. Additionally, the method includes applying one or more voltage cycles to the one or more electrodes to form a coating on the one or more electrodes. The coating includes a polyaniline-containing material.

Further, one or more aspects disclosed relate to a method comprising applying a voltage to an electrode disposed on a surface of a reaction vessel of an oligonucleotide synthesizer apparatus. The electrode has a coating that includes a polyaniline-containing material and a plurality of intermediate oligonucleotide chains are coupled to the electrode. The method also includes, responsive to the voltage being applied to the electrode, causing a protecting group coupled to a terminal nucleotide of an intermediate oligonucleotide chain of the plurality of intermediate oligonucleotide chains to separate from the terminal nucleotide and form an unprotected intermediate oligonucleotide chain.

### BRIEF DESCRIPTION OF THE FIGURES

The drawings illustrate generally, by way of example, but not by way of limitation, various aspects of the present invention.

FIG. 1 is a diagram showing an environment to produce a polyaniline coated electrode, in accordance with one or more implementations.

FIG. 2 is a diagram showing an environment to produce oligonucleotides using polyaniline coated electrodes, in accordance with one or more implementations.

FIG. 3 is diagram of an architecture to encode data using oligonucleotides synthesized using polyaniline coated electrodes, in accordance with one or more implementations.

FIG. 4 is a flow diagram of an example process to produce a polyaniline coated electrode, in accordance with one or more implementations.

FIG. 5 is a flow diagram of an example process to synthesize oligonucleotides using polyaniline coated electrodes, in accordance with one or more implementations.

FIG. 6 shows the setup of electrodes coupled to a potentiometer and contacting a coating solution in addition to the creation of the polyaniline-containing film after performing a number of voltage cycles.

FIG. 7 shows images of electrodes coated with a polyaniline-containing film after applying 5 voltage cycles and after applying 15 voltage cycles at both 1× magnification and 5× magnification.

FIG. 8 shows a graphic that indicates the increase in current density as the number of voltage cycles applied to the electrode went from 0 cycles to 5 cycles to 15 cycles.

FIG. 9 shows an apparatus to synthesize oligonucleotides using electrodes coated with a polyaniline-containing film.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the inventive subject matter, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O)), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some aspects, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some aspects, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)—CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some aspects, the cycloalkyl group can have 3 to about 8-12 ring members, whereas in other aspects the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some aspects, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations.

The terms “polynucleotide”, “nucleic acid”, “nucleic acid molecule”, “polynucleotide molecule”, or “oligonucleotide” refer to a linear polymer of nucleotides or nucleosides joined by internucleosidic linkages. A polynucleotide can comprise at least three nucleotides or three nucleosides Oligonucleotides often range in size from a few monomeric units, e.g., 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes adenosine or deoxyadenosine, “C” denotes cytosine or deoxycytidine, “G” denotes guanine or deoxyguanosine, and “T” denotes thymine or deoxythymidine, unless otherwise noted. The letters A, C, G, and T may be used to refer to the bases themselves, to nucleosides, or to nucleotides comprising the bases, as is standard in the art.

As used herein, “deoxyribonucleic acid” or “DNA” refers to a natural or modified polynucleotide which has a hydrogen group at the 2′-position of the sugar moiety. DNA can include a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA can include a chain of nucleotides comprising four types of nucleotides: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data”, “nucleic acid sequencing information”, “sequence information”, “nucleic acid sequence”, “nucleotide sequence”, “sequencing read”, or “nucleic acid sequencing read” denotes any information or data that is indicative of the order and identity of the nucleotide bases (e.g., adenine, guanine, cytosine, and thymine or uracil) in a molecule (e.g., oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

The terms, “binary data”, “digital information”, or “digital data” refers to data encoded using the standard binary code, or a base 2 {0, 1} alphabet, data encoded using a hexadecimal base 16 alphabet, data encoded using the base 10 {0-9} alphabet, data encoded using ASCII characters, or data encoded using any other discrete alphabet of symbols or characters in a linear encoding fashion.

In one or more examples, the synthetic production of biopolymers can take place by joining monomer units in an electrochemical process. For example, applying a voltage to an array of electrodes that are immersed in a solution can cause the addition of monomer units to a growing molecular scaffold. To illustrate, the molecular scaffold can include linker components and, optionally, one or more monomer units, where the linker components can be coupled to the electrodes. In one or more additional examples, the linker components can be coupled to one or more additional substrates to produce oligonucleotides comprised of a number of monomer units. Applying a voltage to the array of electrodes can cause one or more chemical reactions to take place that can prepare the molecular scaffold to add new monomer units. In at least some examples, the biopolymers produced using synthetic processes can have fewer nucleotides than biopolymers produced using natural processes within an organism. In one or more illustrative examples, biopolymers produced using synthetic processes can comprise no greater than 1,000 monomer units and biopolymers produced by natural processes within an organism can produce biopolymers having at least thousands, up to tens of thousands, up to millions of monomer units.

In one or more illustrative examples, nucleic acids can be synthesized by adding nucleotides to a molecular scaffold that comprises an intermediate oligonucleotide chain. For example, deoxyribonucleic acid (DNA) molecules and ribonucleic acid (RNA) molecules can be formed by coupling monomer units comprised of adenine (A), guanine (G), cytosine (C), and thymine (T), in the case of DNA, or A, G, C, and uracil (U), in the case of RNA. Typically, synthetic polynucleotides are produced according to a number of predetermined sequences. The predetermined sequences can correspond to at least one of the primers used in polynucleotide sequencing operations. The predetermined sequences can also correspond to identifiers that can be used to identify molecules and/or families of molecules after the sequencing process has been performed. In various examples, the predetermined sequences can correspond to digital data that has been encoded within sequences of oligonucleotides. In one or more additional examples, the predetermined sequences can correspond to nucleic acids that can be used in one or more diagnostic techniques. In one or more further examples, the predetermined sequences can correspond to nucleic acids that can be used to deliver one or more therapeutics to patients. In still other examples, the predetermined sequences can be used in one or more gene therapy procedures. The predetermined sequences can also be used in one or more biomedical procedures.

In at least some examples, the coupling of nucleotides can include successively adding nucleotides to an intermediate oligonucleotide chain until a completed oligonucleotide is produced having a sequence of bases that corresponds to the predetermined sequence. In one or more instances, the addition of nucleotides can be controlled such that a given nucleotide is added to one or more specified intermediate oligonucleotide chains. For example, during one round of synthesizing oligonucleotides, the nucleotide adenine can be added to a number of intermediate oligonucleotide chains for which adenine is the next nucleotide in the predetermined sequence. The process can continue with another round of oligonucleotide synthesis causing thymine nucleotides to be added to a number of intermediate oligonucleotide chains for which thymine is the next nucleotide in the predetermined sequence. That is, nucleotides can be selectively added to intermediate oligonucleotide chains according to the predetermined oligonucleotide sequences.

In one or more examples, nucleotides can be selectively added to intermediate oligonucleotide sequences using blocking groups. Blocking groups can prevent new nucleotides from being added to intermediate oligonucleotide sequences by preventing reactions between reactive groups of free nucleotides and reactive groups of the intermediate oligonucleotide chains. The blocking groups can include trityl groups that protect the 5′-hydroxyl group of the terminal nucleotides of a growing oligonucleotide chain. In one or more illustrative examples, the trityl group can include 4,4′-dimethoxytrityl (DMT).

In various examples, oligonucleotides can include reactive groups that, under appropriate conditions, can result in the continuous addition of nucleotides to an intermediate oligonucleotide chain. To illustrate, oligonucleotides can be synthesized using nucleoside phosphoramidite building blocks. Without the blocking groups present on one or more reactive moieties of the terminal nucleotide of the intermediate oligonucleotide chain, the nucleoside phosphoramidite building blocks may be added in a greater number or to unwanted reactive sites than desired. Nucleotides can be added to the molecular scaffold by causing removal of at least one of the blocking groups to enable the reactive moieties of the nucleoside phosphoramidite to react with a reactive moiety of the terminal nucleotide of the intermediate oligonucleotide chain. In one or more examples, the blocking groups can be removed in an acidic environment. For example, the bonds between DMT blocking groups and the nucleoside reactive moieties can be broken down in an acidic environment. The removal of the blocking groups frees the 5′-OH of the terminal nucleotide of the intermediate oligonucleotide chain to react with a free nucleoside building block and bind the new nucleoside to the molecular scaffold.

After a nucleotide has been added to intermediate oligonucleotide chains, one or more washing operations can be performed to remove free nucleoside phosphoramidite building blocks from the presence of the intermediate oligonucleotide chains. Unreacted 5′-OH groups can be capped through an acetylation process to prevent the elongation of the intermediate oligonucleotide chains that may result in oligonucleotides with unwanted modifications, such as deletion mutations. The phosphite tri-ester bond between the scaffold molecule and the added nucleoside can be stabilized through oxidation to create a phosphate tri-ester bond. A number of cycles of removal of the N-terminal deprotection groups and addition of nucleoside phosphoramidites can be repeated. In this way, nucleoside phosphoramidite building blocks can be sequentially added to a molecular scaffold that includes one or more nucleotides and, optionally, a linker group.

In some scenarios, the deblocking step is performed using an acidic solution that removes the blocking groups from the terminal nucleotides of the intermediate oligonucleotide chains. In implementations described herein, deblocking is performed using electrochemical processes. For example, an array of electrodes can be provided in an oligonucleotide synthesis apparatus. Linker molecules can be coupled to the array of electrodes where the linker molecules comprise a foundation on which to build oligonucleotides. In addition, a synthesis solution can be added to the oligonucleotide synthesis apparatus that includes nucleoside phosphoramidite building blocks. Nucleoside phosphoramidites can be sequentially added to the growing oligonucleotide chain by applying a voltage to at least a portion of the electrodes Applying a voltage to electrodes of the array can cause protonization of the synthesis solution around the electrodes to take place that produces an acidic environment with respect to the intermediate oligonucleotide chain coupled to the electrodes. Producing an acidic environment around the oligonucleotide chains coupled to the electrodes can cause blocking groups of the terminal nucleotides to be removed and enable the additional of nucleoside phosphoramidites to the 5′ ends of the terminal nucleotides of the growing intermediate oligonucleotide chains.

In one or more examples, oligonucleotides can be synthesized using an oligonucleotide synthesizing apparatus that includes an array of electrodes coated with a polyaniline-containing material. The polyaniline-containing material can be disposed as one or more layers on one or more surfaces of the electrodes. An electrochemical deposition process can be performed to form one or more layers of a polyaniline-containing material on one or more surfaces of the electrodes. The electrochemical deposition process can be performed by applying a voltage to the electrodes with the electrodes being immersed in a synthesis solution that includes an amount of at least one acid and an amount of aniline monomers. The voltage can cycle between a lower threshold and an upper threshold at a given rate. As the number of voltage cycles increases, a thickness of the polyaniline-containing material on the electrodes can also increase. The manner in which the voltage is applied to the electrodes, such as the voltage values, the voltage rate, and the number of cycles, can cause a conductive form of a polyaniline-containing material to be deposited on the electrodes.

The use of electrodes that are coated with a polyaniline-containing material to perform deblocking of nucleotides of an intermediate oligonucleotide chain can result in greater protonization of the solution proximate to the electrodes. This can lead to local acidification of the solution proximate to the electrodes without any physical separation between the different intermediate oligonucleotide chains. In this way, multiple nucleotides can be synthesized in the same oligonucleotide synthesis apparatus by activating a pattern of electrodes according to the nucleotides that are to be added to a growing oligonucleotide chain. For example, a first portion of intermediate oligonucleotide chains can have an adenine as the next nucleotide to add to the sequence and a second portion of the intermediate oligonucleotide chains can have a guanine as the next nucleotide to add to the sequence. In these scenarios, when a solution including deoxyadenosine phosphoramidites is added to the oligonucleotide synthesis apparatus, first electrodes that correspond to the first intermediate oligonucleotide chains are activated to deblock the terminal nucleotides of the first intermediate oligonucleotide chains and add the deoxyadenosine phosphoramidites to the respective terminal nucleotides. After capping and oxidation, a solution including deoxyguanosine phosphoramidites can be added to the oligonucleotide synthesis apparatus and second electrodes that correspond to the second intermediate oligonucleotide chains can be activated. The activation of the second electrodes can cause deblocking of the terminal nucleotide of the second intermediate oligonucleotide chains coupled to the second electrodes and the deoxyguanosine phosphoramidites can be added to the deblocked second intermediate oligonucleotide chains.

In addition to enabling the synthesis of different oligonucleotides within the same oligonucleotide synthesis apparatus, the use of electrodes coated with a polyaniline-containing material can increase the speed at which the oligonucleotide synthesis process takes place. For example, the use of electrodes coated with a polyaniline-containing material can produce a greater number of protons proximate to the activated electrodes than existing electrochemical oligonucleotide synthesis systems. The increased amounts of protons generated in relation to the activated electrodes can speed up the deblocking of intermediate oligonucleotides and result in less time being taken to synthesize oligonucleotides.

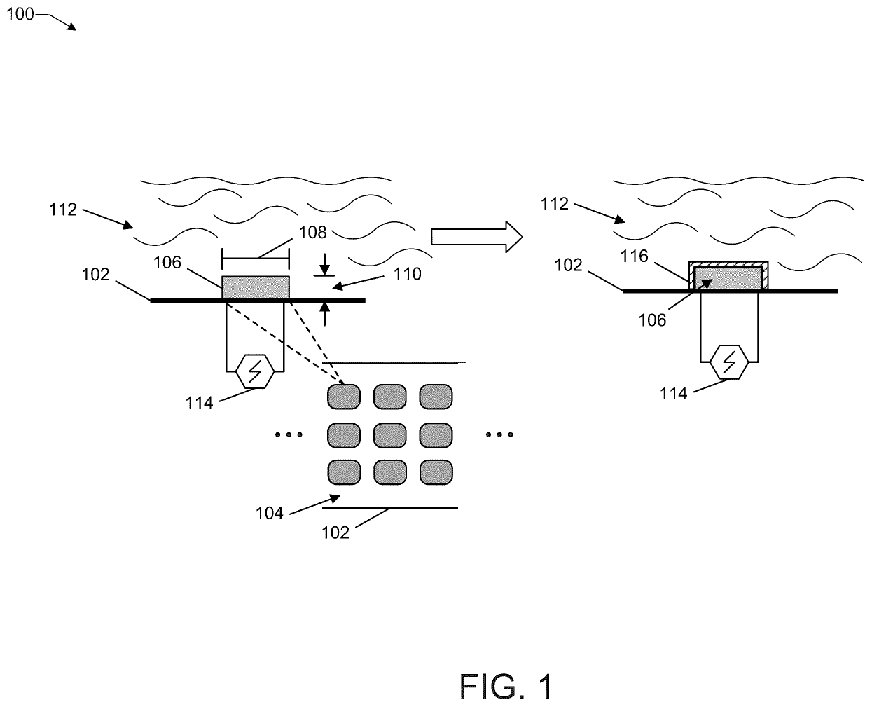

FIG. 1 is a diagram showing an environment 100 to produce a polyaniline coated electrode, in accordance with one or more implementations. The environment 100 can include a substrate portion 102 that is part of an oligonucleotide synthesis apparatus. The substrate portion 102 can include a portion of a surface of an oligonucleotide synthesis apparatus. For example, the substrate portion 102 can include at least a portion of a bottom surface of a reaction vessel of an oligonucleotide synthesis apparatus. In one or more additional examples, the substrate portion 102 can include at least a portion of a surface of a side wall of a reaction vessel of an oligonucleotide synthesis apparatus. The substrate portion 102 can be comprised of one or more metallic materials. In one or more further examples, the substrate portion 102 can be comprised of one or more non-metallic materials. To illustrate, the substrate portion 102 can be comprised of one or more ceramic materials. In one or more additional illustrative examples, the substrate portion 102 can be comprised of one or more glass materials. In one or more further illustrative examples, the substrate portion 102 can be comprised of one or more polymeric materials. In still other examples, the substrate portion 102 can include one or more semiconducting materials, such as silicon.

An electrode array 104 can be disposed on the substrate portion 102. In at least some examples, the electrode array 104 can be comprised of at least tens of electrodes, hundreds of electrodes, thousands of electrodes, up to tens of thousands of electrodes, or more. For example, a representative electrode 106 can be disposed on the substrate portion 102. In one or more examples, the electrode 106 can be comprised of one or more conductive materials. In various examples, the electrode 106 can be comprised of one or more metallic materials. To illustrate, the electrode 106 can include a gold-containing material. In one or more additional illustrative examples, the electrode 106 can include a stainless-steel containing material. In one or more further illustrative examples, the electrode 106 can be comprised of one or more carbon-containing conductive materials. Additionally, the electrode 106 can be comprised of one or more silicon-containing conductive materials. In still other examples, the electrode 106 can be comprised of one or more conductive polymeric materials. Further, the electrode 106 can be comprised of one or more conductive ceramic materials. In one or more scenarios, the electrode 106 can be comprised of one or more indium-tin-oxide materials. In one or more instances, the electrode 106 can be comprised of one or more fluorine-doped tin oxide materials. In various other situations, the electrode 106 can be comprised of one or more semiconductor materials. In at least some examples, the electrode 106 can comprise an anode. In scenarios where the electrode 106 comprises an anode, although not shown in the illustrative example of FIG. 1, the oligonucleotide synthesis apparatus can include a corresponding cathode.

In various examples, the electrode 106 can have a rectangular shape. In one or more additional examples, the electrode 106 can have a circular shape. In at least some examples, the electrode 106 can have a dimension 108, such as at least one of a width, a length, or a diameter no greater than 500 micrometers (μm), no greater than 250 μm, no greater than 100 μm, no greater than 50 μm, no greater than 25 μm, or no greater than 10 μm. In one or more further examples, the electrode 106 can have a dimension 108, such as at least one of a width, a length, or a diameter of no greater than 500 nanometers (nm), no greater than 250 nm, no greater than 100 nm, no greater than 50 nm, no greater than 25 nm, or no greater than 10 nm. In one or more illustrative examples, the electrode 106 can have a dimension 108, such as at least one of a width, a length, or a diameter from about 5 nm to about 100 nm, from about 50 nm to about 250 nm, from about 500 nm to about 2000 nm, from about 1 μm to about 100 μm, from about 25 μm to about 250 μm, or from about 50 μm to about 500 μm.

In still other examples, the electrode 106 can have a dimension 108, such as at least one of a width, a length, or a diameter that is larger. For example, the electrode 106 can have a dimension 108 on the order of one or more micrometers (mm) to tens of millimeters. To illustrate, the electrode 106 can have a dimension 108 no greater than about 200 mm, no greater than about 100 mm, no greater than about 80 mm, no greater than about 50 mm, no greater than about 30 mm, no greater than about 10 mm, no greater than about 5 mm, or no greater than about 1 mm. The electrode 106 can also have a dimension 108 of at least 1 μm, at least about 5 μm, at least about 10 μm, at least about 20 μm, at least about 50 μm, at least about 80 μm, at least about 100 μm, at least about 200 μm, at least about 500 μm, or at least about 800 μm. In various examples, the electrode 106 can have a dimension 108 from about 1 μm to about 100 μm, from about 100 μm to about 200 mm, from about 1 μm to about 500 μm, from about 100 μm to 1 mm, from about 500 μm to about 50 mm, from about 1 mm to about 200 mm, from about 50 mm to about 200 mm, or from about 1 μm to about 200 mm.

The electrode 106 can also have a thickness 110. The thickness 110 can be at least 2000 nm, at least 1000 nm, at least 500 nm, at least 250 nm, at least 100 nm, at least 50 nm, at least 25 nm, at least 10 nm, or at least 5 nm. In one or more illustrative examples, the thickness 110 can be from about 1 nm to about 2000 nm, from about 1 nm to about 50 nm, from about 5 nm to about 100 nm, from about 25 nm to about 100 nm, from about 100 nm to about 250 nm, from about 500 nm to about 1000 nm, or from about 250 nm to about 500 nm.

The electrode 106 can be immersed in a coating solution 112. In one or more examples, the electrode 106 can be immersed in a coating solution 112 that is disposed in a reaction vessel of an oligonucleotide synthesis apparatus. The coating solution 112 can be used to generate a coating that is disposed over at least a portion of the electrode 106. The coating solution 112 can be an aqueous solution that comprises at least about 50% by weight H₂O, at least about 55% by weight H₂O, at least about 60% by weight H₂O, at least about 65% by weight H₂O, at least about 70% by weight H₂O, at least about 75% by weight H₂O at least about 80% by weight H₂O, at least about 85% by weight H₂O, or at least about 90% by weight H₂O.

In one or more examples, the coating solution 112 can include a solvent in addition to H₂O. For example, the coating solution 112 can include a polar aprotic solvent. In one or more additional examples, the coating solution can comprise an amount of acetonitrile. In one or more further examples, the coating solution 112 can be free of H₂O. In at least some examples, the coating solution 112 can comprise at least about 10% by weight acetonitrile, at least about 20% by weight acetonitrile, at least about 30% by weight acetonitrile, at least about 40% by weight acetonitrile, at least about 50% by weight acetonitrile, at least about 60% by weight acetonitrile, at least about 70% by weight acetonitrile, at least about 80% by weight acetonitrile, or at least about 90% by weight acetonitrile.

The coating solution 112 can also have an acid content that is comprised of one or more acids. In one or more examples, the coating solution 112 can have a concentration of acid content of at least 0.005 M, at least 0.01 M, at least 0.02 M, at least 0.05 M, at least 0.1 M, at least 0.15 M, at least 0.2 M, at least 0.25 M, or at least 0.3 M. In one or more illustrative examples, the coating solution 112 can have a concentration of acid content from about 0.005 M to about 0.1 M, from about 0.01 M to about 0.1 M, from about 0.05 M to about 0.25 M, from about 0.1 M to about 0.3 M, or from about 0.15 M to about 0.3 M. In various examples, the acid content of the coating solution 112 can be comprised of H₂SO₄. In one or more additional examples, the acid content of the coating solution 112 can be comprised of HCl. In one or more further examples, the acid content of the coating solution 112 can be comprised of a combination of H₂SO₄ and HCl. In at least some scenarios, a minimum concentration of acid content is present in the coating solution 112 in order to promote polymerization of monomer units that are also present in the coating solution 112.

Additionally, the coating solution 112 can have monomer content. The monomer content includes monomer units that can be used to form a coating on the electrode 106 that comprises at least one polymer comprised of the monomer units. In one or more examples, the monomer content can include aniline monomers. The aniline monomers can have the following composition according to Formula I:

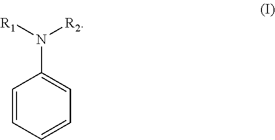

In Formula I, R₁ and R₂ can individually include H or a substituted or unsubstituted chain including from 1 carbon atom to 8 carbon atoms. In one or more illustrative examples, R₁ and R₂ can individually include H or one or more substituted or unsubstituted alkane groups. In one or more additional examples, R₁ and R₂ can include one or more methyl groups, one or more ethyl groups, one or more propyl groups, one or more butyl groups, one or more pentyl groups, one or more hexyl groups, one or more heptyl groups, or one or more octyl groups. In still other examples, the aniline monomer can include thioaniline.

In various examples, the coating solution 112 can have a concentration of monomer content of at least 0.05 M, at least 0.1 M, at least 0.2 M, at least 0.25 M, at least 0.3 M, at least 0.35 M, at least 0.4 M, at least 0.45 M, at least 0.5 M, at least 0.55 M, at least 0.6 M, at least 0.65 M, at least 0.7 M, at least 0.75 M, or at least 0.8 M. In one or more illustrative examples, the coating solution 112 can have a concentration of monomer content from about 0.05 M to about 0.5 M, from about 0.1 M to about 0.8 M, from about 0.25 M to about 0.75 M, from about 0.4 M to about 0.6 M, from about 0.2 M to about 0.7 M, or from about 0.4 M to about 0.8 M.

The electrode 106 can be coupled to a voltage source 114. Although the illustrative example of FIG. 1 shows the voltage source 114 coupled to a single electrode 106, the voltage source 114 can be coupled to a plurality of electrodes of the electrode array 104. Applying a voltage to the electrode 106 using the voltage source 114 when the electrode 106 is immersed in the coating solution 112 can cause a coating 116 to be formed on one or more surfaces of the electrode 106. In one or more examples, the coating 116 can be formed on at least one of a top surface of the electrode 106 or one or more side surfaces of the electrode 106.

In one or more examples, the voltage applied to the electrode 106 can be cycled between a lower threshold voltage to an upper threshold voltage at a voltage cycle rate. The lower threshold voltage can be at least −0.8 volts (V), at least −0.6 V, at least −0.4 V, at least −0.2 V, at least 0 V, at least 0.2 V, at least 0.4 V, or at least 0.6 V. In addition, the upper threshold voltage can be no greater than no greater than 5 V, no greater than 4.8 V, no greater than 4.5 V, no greater than 4.2 V, no greater than 4 V, no greater than 3.8 V, no greater than 3.5 V, no greater than 3.2 V, no greater than 3 V, no greater than 2.8 V, no greater than 2.5 V, no greater than 2.2 V, no greater than 2 V, no greater than 1.8 V, no greater than 1.6 V, no greater than 1.4 V, no greater than 1.2 V, no greater than 1 V, no greater than 0.8 V, or no greater than 0.6 V. In one or more illustrative examples, a voltage cycle applied to the electrode 106 to form the coating 116 can be from about −0.8 V to about 2.2 V, from about −0.6 V to about 2 V, from about −0.4 V too about 1.8V, from about −0.2 V to about 1.6 V, from about −0.4 V to about 1.4 V, from about −0.6 V to about 5 V, from about 2 V to about 5 V, from about 0 V to about 2 V, from about 1 V to about 4 V, or from about −0.2 V to about 1.2 V.

The voltage cycle rate applied by the voltage source 114 to the electrode 106 to produce the coating 116 can be at least 0.01 millivolts (mV)/second (s), at least 0.02 mV/s, at least 0.03 mV/s, at least 0.04 m V/s, at least 0.05 mV/s, at least 0.06 mV/s, at least 0.07 mV/s, at least 0.08 mV/s, at least 0.09 mV/s, or at least 0.1 mV/s. In one or more examples, the voltage cycle rate applied by the voltage source 114 to the electrode 106 to produce the coating 116 can no greater than 0.2 mV/s, no greater than 0.18 mV/s, no greater than 0.16 mV/s, no greater than 0.14 mV/s, no greater than 0.12 m V/s, no greater than 0.1 mV/s, no greater than 0.08 mV/s, or no greater than 0.06 mV/s. In one or more illustrative examples, the voltage cycle rate applied by the voltage source 114 to the electrode 106 to produce the coating 116 can be from about 0.01 m V/s to about 0.2 mV/s, from about 0.02 m V/s to about 0.18 mV/s, from about 0.03 m V/s to about 0.16 m V/s, from about 0.04 m V/s to about 0.14 mV/s, from about 0.05 m V/s to about 0.12 m V/s, from about 0.04 m V/s to about 0.1 m V/s, or from about 0.02 m V/s to about 0.08 m V/s.

In at least some examples, a voltage cycle can include applying a substantially constant voltage over a period of time. For example, a voltage cycle can include applying a voltage over a period of time from about 0.2 V to about 5 V, from about 0.5 V to about 4 V, from about 1 V to about 3 V, from about 0.5 V to about 3 V, or from about 0.2 V to about 2 V. In one or more examples, the voltage cycle can have duration from about 30 seconds to about 5 minutes, from about 1 minute to about 4 minutes, from about 20 seconds to about 3 minutes, or from about 40 seconds to about 2 minutes.

In various examples, a number of voltage cycles can be implemented to cause the coating 116 to be formed on the electrode 106. A voltage cycle can comprise causing the voltage source 114 to increase the voltage applied to the electrode 106 from a lower threshold to an upper threshold at a voltage cycle rate and then decrease the voltage applied to the electrode 106 from the upper threshold back to the lower threshold. In various examples, as the number of voltage cycles applied to the electrode 106 by the voltage source 114 increases, the thickness 110 of the coating 116 can also increase. In one or more examples, the voltage source 114 can implement at least 5 voltage cycles to form the coating 116, at least 8 cycles to form the coating 116, at least 10 voltage cycles to form the coating 116, at least 12 voltage cycles to form the coating 116, at least 15 voltage cycles to form the coating 116, at least 18 voltage cycles to form the coating 116, at least 20 voltage cycles to form the coating 116, at least 22 voltage cycles to form the coating 116, or at least 25 voltage cycles to form the coating 116. In one or more illustrative examples, the voltage source 114 can implement from 5 voltage cycles to 25 voltage cycles to form the coating 116, from 8 voltage cycles to 22 voltage cycles to form the coating 116, from 10 voltage cycles to 20 voltage cycles to form the coating 116, from 15 voltage cycles to 25 voltage cycles to form the coating 116, or from 5 voltage cycles to 15 voltage cycles to form the coating 116.

In one or more scenarios, applying too few voltage cycles to the electrode 106 can result in a coating 116 having a thickness that is not sufficient to effectively cause deblocking of protecting groups and form oligonucleotides using the electrode 106. For example, in situations where a thickness of the coating 116 is less than a lower threshold thickness, the electrode array 104 may be unable to produce a sufficient amount of protons to cause deblocking and produce oligonucleotides. In one or more additional examples, applying too many voltage cycles to the electrode 106 can result in a coating 116 that is too thick to effectively cause deblocking of protecting groups and form oligonucleotides using the electrode 106. To illustrate, in scenarios where the coating 116 has a thickness greater than an upper threshold, during synthesis of oligonucleotides, portions of the coating 116 may detach from the coating 116 and cause contamination of the synthesis solution used during the production of oligonucleotides. In one or more illustrative examples, the coating 116 can have a thickness 110 of at least 5 nm, at least 10 nm, at least 20 nm, at least 30 nm, at least 40 nm, at least 50 nm, at least 75 nm, or at least 100 nm. In one or more additional illustrative examples, the coating 116 can have a thickness 110 from about 5 nm to about 100 nm, from about 10 nm to about 75 nm, from about 25 nm to 50 nm, from about 25 nm to about 75 nm, or from about 10 nm to about 50 nm. In at least some example, the thickness 110 of the coating 116 can correspond to an average thickness of the coating 116 disposed on the electrode 106.

In one or more illustrative examples, the coating 116 can be formed using an aqueous solution having an acid content from about 0.05 M H₂SO₄ to about 0.2 M H₂SO₄ or from about 0.05 M HCl to about 0.2 M HCl and a monomer content from about 0.3 M aniline to about 0.6 M aniline and applying from 10 voltage cycles to 20 voltage cycles using the voltage source 114, where the individual voltage cycles have a lower threshold from about −0.4 V to 0 V and an upper threshold from about 0.8 V to about 1.4 V at a voltage cycle rate from 0.02 V/s to about 0.08 m V/s.

In at least some examples, the coating 116 can comprise a polyaniline-containing material. The polyaniline-containing material can include a form of polyaniline that corresponds to an emeraldine salt. In various examples, the coating solution 112 can include one or more electrolytes that can be used to produce the emeraldine salt. In one or more examples, the coating solution 112 can include a lithium-containing electrolyte. In one or more additional examples, the coating solution 112 can include a chlorine-containing electrolyte. In one or more illustrative examples, the coating solution 112 can include lithium perchlorate and the emeraldine salt can include a salt comprising polyaniline and a perchlorate ion. In one or more additional illustrative examples, the coating solution 112 can include a salt comprising polyaniline and one or more persulfates ions. In one or more further illustrative examples, the coating solution 112 can include a salt comprising polyaniline and one or more peroxyphosphate ions. In still other examples, the coating solution 112 can include a salt comprising polyaniline and one or more ions derived from one or more peroxyacids.

In various examples, the voltages applied to the electrode 106 by the voltage source 114, the number of voltage cycles implemented using the voltage source 114, the acid content of the coating solution 112, and the monomer content of the coating solution 112 can produce the coating 116, such that the coating 116 comprises a conductive form of polyaniline. In one or more examples, the coating 116 can comprise a polyaniline-containing material having according to Formula II:

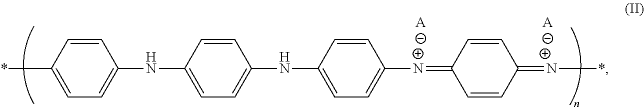

where A is a salt complexed with a portion of a polyaniline chain.

In at least some examples, the coating 116 can be comprised of a polyaniline-containing material having an electrical conductivity at 20° C. of no greater than 6.0×10⁶ siemens per meter (S/m), no greater than 5×10⁶ siemens per meter, no greater than 4×10⁶ siemens per meter, or no greater than 3×10⁶ S/m. Additionally, the coating 116 can be comprised of a polyaniline-containing material having an electrical conductivity at 20° C. of at least 2×10⁵ S/m, at least 3×10⁵ S/m, at least 4×10⁵ S/m, at least 5×10⁵ S/m, at least 6×10⁵ S/m, at least 7×10⁵ S/m, at least 8×10⁵S/m, at least 9×10⁵ S/m, or at least 1×10⁶ S/m. In one or more illustrative examples, the coating 116 can be comprised of a polyaniline-containing material having an electrical conductivity at 20° C. from about 2×10⁵ S/m to about 6×10⁶ S/m, from about 2×10⁵ S/m to about 1×10⁶ S/m, from about 5×10⁵ S/m to about 4×10⁶ S/m, from about 8×10⁵ S/m to about 4×10⁶ S/m, or from about 1×10⁶ S/m to about 6×10⁶ S/m.

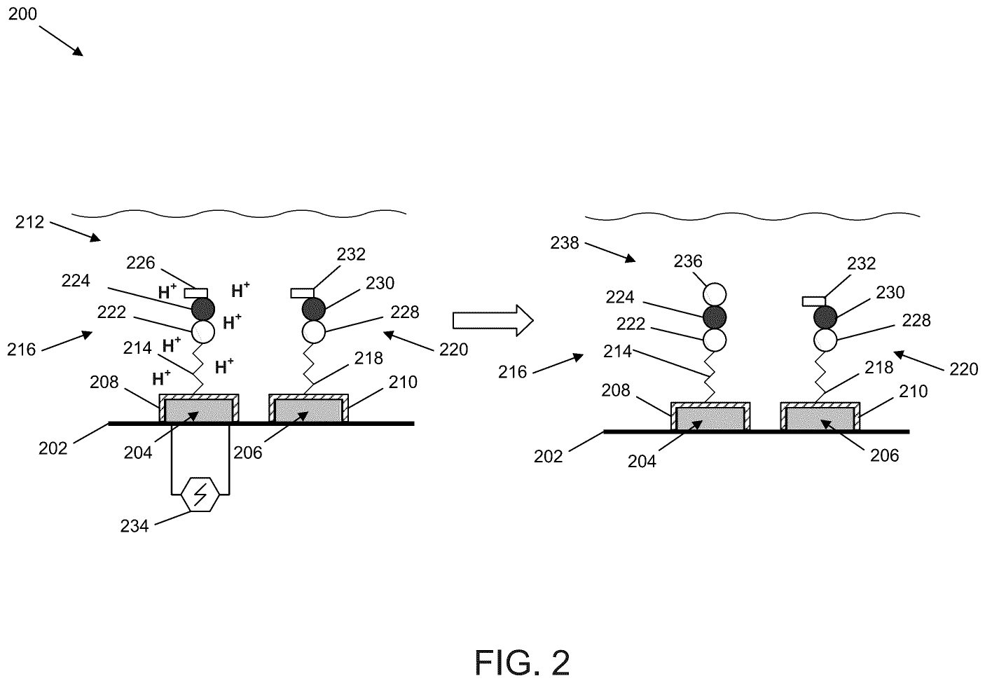

FIG. 2 is a diagram showing an environment 200 to produce oligonucleotides using polyaniline coated electrodes, in accordance with one or more implementations. The environment 200 can comprise at least a portion of an oligonucleotide synthesis apparatus. For example, the environment 200 can comprise at least a portion of a reaction vessel of an oligonucleotide synthesis apparatus. The oligonucleotide synthesis apparatus can include a substrate 202 on which an array of electrodes is disposed. The array of electrodes can include a plurality of electrodes, such as a first electrode 204 and a second electrode 206. In one or more examples, the plurality of electrodes included in the array of electrodes can be arranged according to a pattern. The first electrode 204 can have a first coating 208 and the second electrode 206 can have a second coating 210. In at least some examples, the substrate 202 can correspond to the substrate portion 102 described with respect to FIG. 1 and the electrodes 204, 206 can correspond to the electrode 106 described with respect to FIG. 1. Additionally, the first coating 208 and the second coating 210 can be formed according to the implementations described in conjunction with forming the coating 116 of FIG. 1. A separation between the first electrode 204 and the second electrode 206 can be from about 0.1 μm to about 500 μm, from about 0.5 μm to about 500 μm, from about 0.8 μm to about 100 μm, from about 5 μm to about 500 μm, from about 100 μm to about 750 μm, from about 50 μm to about 1000 μm, or from about 500 μm to about 1000 μm.

The electrodes 204, 206 can be disposed in a reaction vessel of an oligonucleotide synthesis apparatus that holds a deblocking solution 212. The deblocking solution 212 can include a number of components that facilitate the removal of protecting groups present on terminal nucleotides of an intermediate oligonucleotide chain. For example, the deblocking solution 212 can comprise one or more quinones. For example, the deblocking solution 212 can include at least one of an amount of benzoquinone or an amount of hydroquinone. Additionally, the deblocking solution 212 can include one or more salts. In one or more illustrative examples, the deblocking solution 212 can include tetrabutylammonium hexafluorophosphate. In one or more further examples, the deblocking solution 212 can include one or more solvents. To illustrate, the deblocking solution 212 can include at least one of an amount of acetonitrile or an amount of an alcohol. Further, the deblocking solution 212 can include one or more amines. To illustrate, the deblocking solution 212 can include N,N-diisopropylethylamine. In at least some illustrative examples, the deblocking solution 212 can comprise one or more solutions described in U.S. Pat. No. 9,267,213.

A number of intermediate oligonucleotide chains can be coupled to the electrodes 204, 206. In one or more examples, the number of intermediate oligonucleotide chains can be coupled to the electrodes 204, 206 using one or more linker molecules. In one or more additional examples, the number of intermediate oligonucleotide chains can be coupled to the electrodes 204, 206 without using a linker molecule. In one or more illustrative examples, the intermediate oligonucleotide chains can include at least a first linker 214 to couple a first chain of nucleotides 216 to the first electrode 204 and a second linker 218 to couple a second string of nucleotides 220 to the second electrode 206. The first linker 214 can be coupled to the first coating 208 on the first electrode 204 and the second linker 218 can be coupled to the second coating 210 on the second electrode 206. In at least some examples, because the first coating 208 and the second coating include an organic polymer, such as a polyaniline, the first linker 214 and the second linker 218 can have an affinity to the first coating 208 and the second coating 210 that is greater than an affinity between the first linker 214 and the second linker 218 with the first electrode 204 and the second electrode 206 absent the first coating 208 and the second coating 210. The first linker 214 can be covalently bonded to functional groups of the polyaniline molecules that are included in the first coating 208 and the second linker 218 can be covalently bonded to functional groups of the polyaniline molecules that are included in the second coating 210.

The first linker 214 and the second linker 218 can include a carbon backbone with one or more first functional groups to attach to the first coating 208 and the second coating 210 and one or more second functional groups to attach to the first nucleotide 222 and the first additional nucleotide 228. The carbon backbone can include at least one of one or more alkyl groups or one or more alkenyl groups. The one or more first functional groups and the one or more second functional groups can include at least one of an ester-containing functional group, an amide-containing functional group, a hydroxyl functional group, or an amine-containing functional group. In one or more illustrative examples, the linkers 214, 218 can be at least one of electrochemically cleavable from the coatings 208, 210; thermally cleavable from the coatings 208, 210; or chemically cleavable from the coatings 208, 210.

In one or more illustrative examples, the linkers 214, 218 can include a functionalized compound according to Formula III:

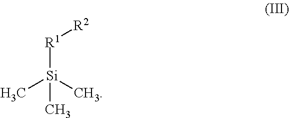

In Formula III, R¹ is selected from the group consisting of a bond or (C₁-C₂₀)hydrocarbyl. R² is selected from the group consisting of —OH and N(R₃)₂. At each instance, R³ is independently selected from the group consisting of —H or substituted or unsubstituted (C₁-C₂₀)hydrocarbyl. In further examples, R¹ is selected from the group consisting of a bond or substituted or unsubstituted (C₂-C₇)hydrocarbyl. In still further examples, R¹ is selected from the group consisting of a bond or substituted or unsubstituted (C₂-C₂₀)alkenyl, substituted or unsubstituted (C₂-C₂₀)aryl, substituted or unsubstituted (C₂-C₂)hydroxyl, substituted or unsubstituted (C₂-C₂₀)alkyl, or substituted or unsubstituted (C₂-C₂₀)cycloalkyl. In at least some examples, linker molecules 214, 218 are a functionalized 3-aminopropyltriethoxysilane or a functionalized trimethylsilanol.

The first chain of nucleotides 216 can include a first nucleotide 222 and a second nucleotide 224. Additionally, a first blocking group 226 can be coupled to a reactive group of the second nucleotide 224. The second chain of nucleotides 220 can include a first additional nucleotide 228 and a second additional nucleotide 230. Further, a second blocking group 232 can be coupled to a reactive group of the second additional nucleotide 230. Although the illustrative example of FIG. 2 shows that the first nucleotide 222 and the first additional nucleotide 228 include a same nucleotide and the second nucleotide 224 and the second additional nucleotides 230 include a same nucleotide, in one or more additional examples, the first additional nucleotide 228 can be different from the first nucleotide 222 and/or the second additional nucleotide 230 can be different from the second nucleotide 224.

In one or more examples, a plurality of oligonucleotides can be synthesized on individual electrodes 204, 206. A density of oligonucleotides coupled to the electrodes 204, 206 can correspond to a number of oligonucleotides coupled to the individual electrodes 204, 206 per an amount of surface area of the individual electrodes 204, 206. For example, an oligonucleotide density for the individual electrodes 204, 206 can include at least 1 oligonucleotide per 100 nm² of surface area of the individual electrode 204, 206; at least 2 oligonucleotides per 100 nm² of surface area of the individual electrodes 204, 206; at least 5 oligonucleotides per 100 nm² of surface area of the individual electrodes 204, 206; at least 10 oligonucleotides per 100 nm² of surface area of the individual electrodes 204, 206; at least 20 oligonucleotides per 100 nm² of surface area of the individual electrodes 204, 206; at least 30 oligonucleotides per 100 nm² of surface area of the individual electrodes 204, 206; at least 40 oligonucleotides per 100 nm² of surface area of the individual electrodes 204, 206; at least 50 oligonucleotides per 100 nm² of surface area of the individual electrodes 204, 206; or at least 100 oligonucleotides per 100 nm² of surface area of the individual electrodes 204, 206. In various examples, an oligonucleotide synthesis apparatus using an array of electrodes that includes electrodes 204, 206 can synthesize at least 2,000 oligonucleotides; at least 5,000 oligonucleotides; at least 10,000 oligonucleotides; at least 20,000 oligonucleotides; at least 30,000 oligonucleotides; at least 50,000 oligonucleotides; at least 100,000 oligonucleotides; at least 250,000 oligonucleotides; at least 500,000 oligonucleotides; at least 750,000 oligonucleotides; at least 1,000,000 oligonucleotides; at least 2,500,000 oligonucleotides; at least 5,000,000 oligonucleotides; at least 10,000,000 oligonucleotides, at least 50,000,000 oligonucleotides, at least 100,000,000 oligonucleotides, at least 200,000,000 oligonucleotides, or at least 500,000,000 oligonucleotides. In one or more illustrative examples, from about 0.1×10⁻¹⁵ M oligonucleotides to about 0.5×10⁻¹⁵ M oligonucleotides, from about 0.8×10⁻¹⁴ M oligonucleotides to about 0.8×10⁻¹⁵ M oligonucleotides, from about 0.5×10⁻¹⁴ M oligonucleotides to about 1.0×10⁻¹⁶ M oligonucleotides, or from about 0.8×10⁻¹⁴ M oligonucleotides to about 0.3×10⁻¹⁵ M oligonucleotides can be synthesized by an individual electrode of the array of electrodes.

The electrodes 204, 206 can be coupled to a voltage source. For example, the first electrode 204 can be coupled to a voltage source 234. Although not shown in the illustrative example of FIG. 2, the voltage source 234 can be coupled to multiple electrodes of an array of electrodes, including electrodes 204, 206. During syntheses of oligonucleotides, the voltage source 234 can apply a voltage to one or more electrodes. To illustrate, the voltage source 234 can apply a voltage to the first electrode 204 to add a third nucleotide 236 to the first chain of nucleotides 216. The voltage applied to the first electrode 204 can be from about 0.1 V to about 5 V, from about 0.3 V to about 4 V, from about 0.5 V to about 3 V, from about 0.1 V to about 2 V, from about 0.5 V to about 1.5 V, from about 2 V to about 4 V, from about 1.5 V to about 2.5 V, or from about 2.5 V to about 3.5 V.

In one or more examples, applying a voltage to the first electrode 204 can cause protons to be generated in the portion of the deblocking solution 212 proximate to the first electrode 204. The localized increase of protons in the space proximate to the first electrode 204 can decrease the pH of the deblocking solution 212 in the region proximate to the first electrode 204. As the portion of the deblocking solution 212 proximate to the first electrode 204 becomes more acidic, the blocking group 226 can be removed from the first chain of nucleotides 216. In one or more illustrative examples, the first coating 208 can undergo oxidation in response to a voltage being applied to the first electrode 204 by the voltage source 234 and function as a proton donor to the deblocking solution 212. In various examples, the first coating 208 can be reduced when the voltage being applied by the voltage source 234 is removed. As a result, the first coating 208 can become a proton acceptor and the pH of the portion of the deblocking solution 212 proximate to the first electrode 204 can increase and become less acidic.

After removal of the first blocking group 226, the voltage source 234 may no longer apply a voltage to the first electrode 204. As a result, the number of protons present in the deblocking solution 212 can decrease. Thus, the protonization and deprotonization of the portion of the deblocking solution 212 proximate to the first electrode 204 is at least a partially reversible process. Additionally, after removal of the first blocking group 226, a third nucleotide 236 can be added to the first chain of nucleotides 216. The third nucleotide 236 can be present in a synthesis solution 238. The synthesis solution 238 can include nucleoside phosphoramidites that correspond to a nucleotide being added next to the first chain of nucleotides 216. For example, the synthesis solution 238 can include nucleoside phosphoramidites that correspond to adenine, thymine, guanine, or cytosine depending on the nucleotide to be added to the first chain of nucleotides 216.

In the illustrative example of FIG. 2, a nucleotide is not added to the second string of nucleotides 220 because the second deblocking group 232 remains attached to the second additional nucleotide 230. The second deblocking group 232 remains attached to the second additional nucleotide 230 due to a voltage being applied to the first electrode 204 and not to the second electrode 206. In this way, a first oligonucleotide having a first sequence can be synthesized using the first electrode 204 and a second oligonucleotide having a second sequence that is different from the first sequence can by synthesized using the second electrode 206. Thus, oligonucleotides having different sequences can be selectively synthesized within a same reaction vessel and in a parallel manner.

In one or more examples, the addition of nucleotides to the first chain of nucleotides 216 and/or the second chain of nucleotides 220 can be performed at a rate that is faster than existing systems. For example, the first coating 208 of the first electrode 204 and the second coating 210 of the second electrode 206 can be more conductive than existing materials to which intermediate oligonucleotide chains are coupled. As a result, an increased number of protons can be generated during the deblocking step and the removal of protecting groups can occur more quickly in reaction vessels that include an array of electrodes overlaid with coatings that correspond to the first coating 208 and the second coating 210 in relation to existing systems. By increasing the speed at which the deblocking operations are performed, the amount of time to implement a cycle to add a nucleotide to an intermediate oligonucleotide chain is reduced with respect to existing systems. In one or more illustrative examples, a cycle of adding a nucleotide to a number of intermediate oligonucleotide chains using an oligonucleotide synthesis apparatus having electrodes overlaid with coatings that correspond to the first coating 208 and the second coating 210 can be performed in no greater than 250 seconds, no greater than 200 seconds, no greater than 180 seconds, no greater than 150 seconds, no greater than 120 seconds, no greater than 100 seconds, or no greater than 80 seconds.

The oligonucleotides synthesized using the electrodes 204, 206 can include at least 5 nucleotides, at least 10 nucleotides, at least 25 nucleotides, at least 50 nucleotides, at least 75 nucleotides, at least 100 nucleotides, at least 150 nucleotides, at least 200 nucleotides, at least 250 nucleotides, at least 300 nucleotides, at least 400 nucleotides, or at least 500 nucleotides.

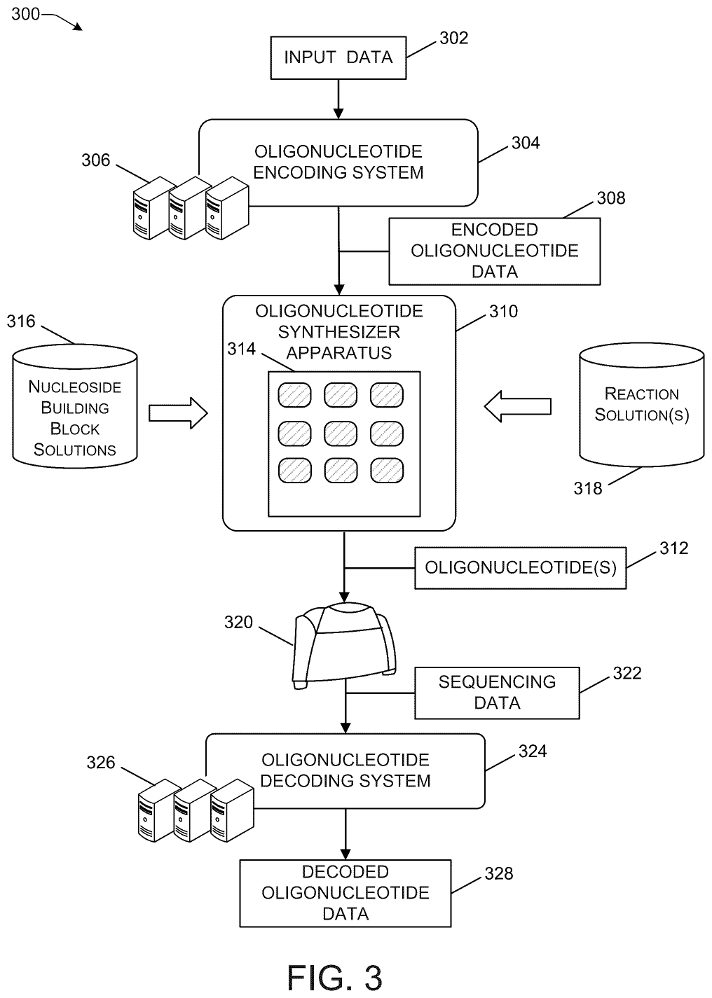

FIG. 3 is diagram of an architecture 300 to encode data using oligonucleotides synthesized using polyaniline coated electrodes, in accordance with one or more implementations. The architecture 300 can include input data 302. The input data 302 can include information that is to be encoded using oligonucleotides. In one or more examples, the input data 302 can include binary data stored in one or more data files. The input data 302 can correspond to digital information stored in one or more documents, one or more databases, one or more applications, one or more media files, or one or more combinations thereof.

The architecture 300 can include an oligonucleotide encoding system 304 that obtains the input data 302 The oligonucleotide encoding system 304 can be implemented by one or more computing devices 306. For example, the one or more computing devices 306 can include at least one of one or more desktop computing devices, one or more mobile computing devices, or one or more server computing device. In various examples, at least a portion of the one or more computing devices 306 can be included in a remote computing environment, such as a cloud computing environment. The oligonucleotide encoding system 304 can analyze the input data 302 and generate oligonucleotide sequences that encode the data. To illustrate, the oligonucleotide encoding system 304 can generate encoded oligonucleotide data 308 that corresponds to the input data 302. The oligonucleotide encoding system 304 can analyze the input data 302 using one or more encoding algorithms to generate the encoded oligonucleotide data 308.

The oligonucleotide sequences included in the encoded oligonucleotide data 308 can correspond to DNA sequences, RNA sequences, or combinations of DNA sequences and RNA sequences. In one or more examples, one or more portions of oligonucleotide sequences included in the encoded oligonucleotide data 308 that correspond to DNA can include sequences represented by the four bases found naturally occurring in DNA: cytosine (C), guanine (G), adenine (A), and thymine (T). One or more portions of oligonucleotide sequences included in the encoded oligonucleotide data 308 that correspond to RNA can include sequences represented by the four bases found naturally occurring in RNA: cytosine (C), guanine (G), adenine (A), and uracil (U) In at least some examples, the oligonucleotide sequences included in the encoded oligonucleotide data 308 can include single stranded oligonucleotide sequences. In one or more additional examples, the oligonucleotide sequences included in the encoded oligonucleotide data 308 can include double stranded sequences. In one or more further examples, the oligonucleotide sequences included in the encoded oligonucleotide data 308 can include a combination of single stranded sequences and double stranded sequences.

The architecture 300 can include an oligonucleotide synthesizer apparatus 310 that synthesizes oligonucleotides 312 based on the encoded oligonucleotide data 308. The oligonucleotide synthesizer apparatus 310 can include an electrode array 314. The electrode array 314 can include a number of electrodes that include a coating that comprises a polyaniline-containing material. In one or more examples, the electrode array 314 can include a number of electrodes that are coated according to the process described with respect to FIG. 1 and the oligonucleotide synthesizer apparatus 310 can operate in a similar manner as described with respect to FIG. 2 to produce the oligonucleotides 312. In various examples, the electrode array 314 can be disposed on one or more surfaces of a reaction vessel of the oligonucleotide synthesize apparatus 310. In one or more illustrative examples, the oligonucleotide synthesizer apparatus 310 can include a number of wells and/or a number of channels with the electrode array 314 disposed within one or more wells of the oligonucleotide synthesizer apparatus 310.

The oligonucleotide synthesizer apparatus 310 can implement synthesis of oligonucleotides using nucleoside phosphoramidites. The synthesis of oligonucleotides by the oligonucleotide synthesizer apparatus 310 can add nucleoside phosphoramidite building blocks in a 3′ to 5′ direction to intermediate oligonucleotide chains. In one or more examples, the nucleoside phosphoramidite building blocks can be added to intermediate oligonucleotide chains in an order that corresponds to the oligonucleotide sequences included in the encoded oligonucleotide data 308. The nucleoside phosphoramidites used to synthesize the oligonucleotides 312 can be included in nucleoside building block solutions 316. In various examples, the nucleoside building block solutions 316 can include multiple solutions with individual solutions including a single nucleoside phosphoramidite. For example, the nucleoside building block solutions 316 can include a first solution that includes deoxyadenosine phosphoramidites, a second solution that includes deoxythymidine phosphoramidites, a third solution that includes deoxyguanosine phosphoramidites, and a fourth solution that includes deoxycytidine phosphoramidites. In situations where the oligonucleotides 312 include RNA sequences, the nucleoside building block solutions 316 can include deoxyuridine phosphoramidites. The nucleoside building block solutions 316 can be solutions that also include at least one of a buffer or a salt.

The architecture 300 can also include reaction solutions 318. The reaction solutions 318 can cause one or more chemical reactions to take place within the oligonucleotide synthesizer apparatus to produce the oligonucleotides 312. For example, the reaction solutions 318 can include a deblocking solution that can include compounds that facilitate the removal of protecting groups from intermediate oligonucleotide chains. Additionally, the reaction solutions 318 can include one or more acetylation solutions to cap unreacted 5′ hydroxyl groups after the addition of nucleoside phosphoramidite building blocks to intermediate oligonucleotide chains. The capping step can prevent intermediate oligonucleotide chains that did not add a nucleoside phosphoramidite building blocks in a synthesis cycle from participating in additional synthesis reactions that may result in a deletion error. The one or more acetylation solutions can include acetic anhydride, N-methyl imidazole, tetrahydrofuran, and pyridine. Further, reaction solutions 318 can include one or more oxidation solutions that can convert the phosphite group of the nucleoside phosphoramidite building blocks to a phosphate group that links the nucleotides of the intermediate oligonucleotide chains. In at least some examples, the one or more oxidation solutions can include iodine, water, and pyridine.

Each cycle of the oligonucleotide synthesis process can cause a single nucleotide to be added to intermediate oligonucleotide chains. The order of the nucleotides added to the intermediate oligonucleotide chains is based on the oligonucleotide sequences included in the encoded oligonucleotide data 308. In one or more illustrative examples, when a next nucleotide to be added to one or more intermediate oligonucleotide chains is adenine, a cycle of the oligonucleotide synthesis process can be performed with a deoxyadenosine phosphoramidite solution and when a next nucleotide to be added to one or more intermediate oligonucleotide chains is thymine, a cycle of the oligonucleotide synthesis process can be performed with a deoxythymidine phosphoramidite solution. Additionally, when a next nucleotide to be added to one or more intermediate oligonucleotide chains is guanine, a cycle of the oligonucleotide synthesis process can be performed with a deoxyguanosine phosphoramidite solution and when a next nucleotide to be added to one or more intermediate oligonucleotide chains is cytosine, a cycle of the oligonucleotide synthesis process can be performed with a deoxycytidine phosphoramidite solution.

After synthesis of oligonucleotide chains on the electrode array 314 is complete and protecting groups are removed, the completed oligonucleotide chains can be cleaved from the electrode array 314 to produce the oligonucleotides 312. In various examples, the completed oligonucleotide chains can be cleaved from the electrode array 314 using one or more chemical processes or one or more electrochemical processes. In one or more examples, the oligonucleotides 312 can be stored under conditions that minimize degradation of the oligonucleotides 312. For example, the oligonucleotides 312 can be stored at temperatures from about −10° C. to −80° C. or from −20° C. to −70° C. in a slightly basic solution. To illustrate, the oligonucleotides 312 can be stored in a solution having a pH from about 7.8 to about 8.2 that includes at least one of Tris(hydroxymethyl)aminomethane hydrochloride or Ethylenediaminetetraacetic acid (EDTA). In one or more additional examples, the oligonucleotides 312 can undergo one or more drying processes and be stored at temperatures from about 10° C. to about 25° C.

The storage of the oligonucleotides 312 in a suitable environment enables the data encoded by the oligonucleotides to be stored until a request is received to retrieve the encoded data. To retrieve data encoded by at least a portion of the oligonucleotides 312, the portion of the oligonucleotides 312 that corresponds to the data being retrieved are provided to a sequencing apparatus 320. The sequencing apparatus 320 can perform one or more sequencing operations to generate sequencing data 322. The sequencing data 322 can include sequence reads that correspond to the nucleotide sequences of at least a portion of the oligonucleotides 312. The sequencing apparatus 320 can implement one or more next generation sequencing techniques. Next generation sequencing techniques can include post-Sanger, high throughput sequencing techniques that sequence millions of nucleotide fragments in parallel. In various examples, the sequencing apparatus 320 can implement other sequencing techniques, such as Sanger sequencing, nanopore sequencing, or single molecular real-time sequencing.

The sequencing data 322 can be analyzed by an oligonucleotide decoding system 324 that is implemented by one or more computing devices 326. The oligonucleotide decoding system 324 can implement one or more computational algorithms to generate decoded oligonucleotide data 328 from the sequencing data 322. For example, the oligonucleotide decoding system 324 can analyze sequence reads to determine at least one of the bits or bytes encoded by the respective sequence reads to produce the decoded oligonucleotide data 328. In one or more examples, the decoded oligonucleotide data 328 can be assembled into a data file that can be read by a computing device. In one or more illustrative examples, the decoded oligonucleotide data 328 can be used to generate a portion of a database that corresponds to at least a portion of the input data 302.

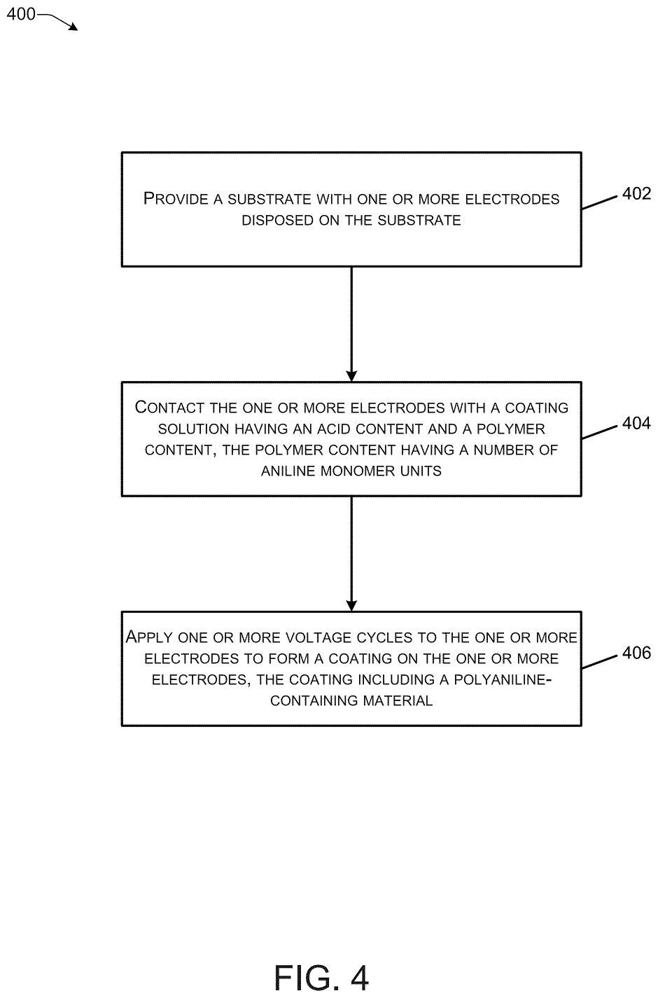

FIG. 4 is a flow diagram of an example process 400 to produce a polyaniline coated electrode, in accordance with one or more implementations. The process 400 includes, at 402, providing a substrate with one or more electrodes disposed on the substrate. The electrodes can include one or more metallic materials. In one or more additional examples, the electrodes can include at least one of one or more ceramic materials or one or more glass materials. In one or more further examples, the electrodes can include one or more polymeric materials, such as one or more organic polymers. In various examples, the electrodes can include one or more inorganic materials. For example, the electrodes can include at least one of carbon or silicon.

In addition, at 404, the process 400 includes contacting the one or more electrodes with a coating solution. The coating solution includes an acid content. The acid content can comprise from about 0.05 M to 0.2 M sulfuric acid (H₂SO₄). The acid content can also comprise from about 0.05 M to 0.2 M hydrochloric acid (HCl) The coating solution also includes a monomer content. The monomer content can have a number of aniline monomer units. In various examples, the monomer content can comprise from about 0.05 M to about 1 M aniline monomer units or from about 0.3 M to about 0.6 M aniline monomer units. In one or more examples, the coating solution can include an aqueous solution having at least 50% by weight H₂O. Additionally, the coating solution can include a polar aprotic solvent. In one or more illustrative examples, the polar aprotic solvent includes acetonitrile. Further, the coating solution can include an electrolyte. To illustrate, the electrolyte can include sodium perchlorate.

Further, the process 400 can include, at 406, applying one or more voltage cycles to the one or more electrodes to form a coating on the one or more electrodes. The coating can include a polyaniline-containing material. Additionally, a voltage cycle of the one or more voltage cycles can include increasing a voltage applied to the one or more electrodes from a lower threshold voltage to an upper threshold voltage at a voltage rate and decreasing the voltage applied to the one or more electrodes from the upper threshold voltage to the lower threshold voltage. In one or more examples, the voltage rate can from about 0.02 millivolts per second (mV/s) to about 0.08 mV/s. Further, in one or more illustrative examples, the lower threshold voltage can be from about −0.6 V to about 0 V and the upper threshold voltage can be from about 0.8 V to about 1.6 V. In various examples, from about 8 voltage cycles to about 20 voltage cycles can be applied to the one or more electrodes to form the coating.

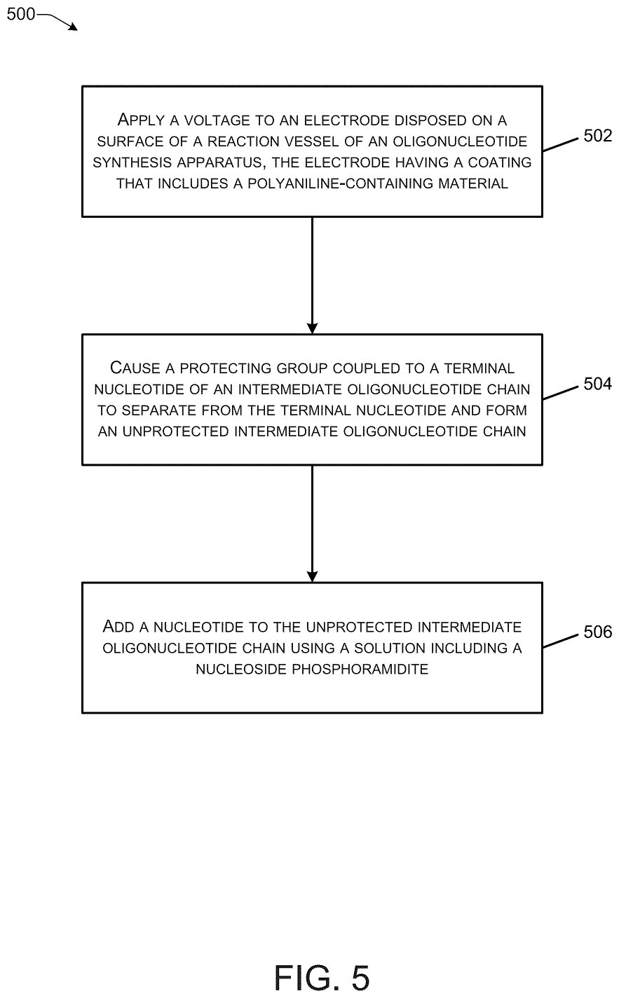

FIG. 5 is a flow diagram of an example process 500 to synthesize oligonucleotides using polyaniline coated electrodes, in accordance with one or more implementations. The process 500 can include, at 502, applying a voltage to an electrode disposed on a surface of a reaction vessel of an oligonucleotide synthesis apparatus. The electrode can have a coating that includes a polyaniline-containing material. In one or more examples, a deblocking solution can disposed in the reaction vessel during the voltage being applied to the electrode. The deblocking solution can include at least one of an amount of benzoquinone or an amount of hydroquinone. The deblocking solution can also include at least one of an amount of tetrabutylammonium hexafluorophosphate, an amount of acetonitrile, or an amount of N,N-diisopropylethylamine.

Additionally, at 504, the process 500 can include causing a protecting group coupled to a terminal nucleotide of an intermediate oligonucleotide chain to separate from the terminal nucleotide and form an unprotected intermediate oligonucleotide chain. The unprotected intermediate oligonucleotide chain can include one or more functional groups of the terminal nucleotide that are exposed and that were previously protected by a protecting group. In one or more examples, responsive to the voltage being applied to the electrode, an oxidation state of the coating can change from a reduced state to an oxidized state to produce protons that decrease the pH of the synthesis solution.

Further, the process 500 can include, at 506, adding a nucleotide to the unprotected intermediate oligonucleotide chain using a solution including a nucleoside phosphoramidite. The nucleotide can be added to the unprotected intermediate oligonucleotide chain by adding a synthesis solution to the reaction vessel. The synthesis solution can include a nucleoside phosphoramidite and the nucleotide phosphoramidite can couple to the unprotected intermediate oligonucleotide chain. In at least some examples, the nucleoside phosphoramidite can correspond to a nucleotide to add to the intermediate oligonucleotide chain in relation to an oligonucleotide sequence that encodes an amount of digital information.

In various examples, the electrode can be included in an array of electrodes disposed on one or more surfaces of the reaction vessel. In one or more examples, the voltage can be applied to a first group of electrodes of the array of electrodes with the first group of electrodes being coupled to a first number of intermediate oligonucleotide chains. A first nucleotide to be added to the first number of intermediate oligonucleotide chains can encode a first set of digital information using a sequence of the first number of intermediate oligonucleotide chains. In these scenarios, the nucleoside phosphoramidite can correspond to the first nucleotide. In one or more examples, subsequent to adding the first nucleotide to the first number of intermediate oligonucleotide chains, an additional voltage is applied to a second group of electrodes to add a second nucleotide to a second number of intermediate oligonucleotide chains attached to the second group of electrodes. The second nucleotide can be added to the second number of intermediate oligonucleotide chains to encode a second set of digital information using an additional sequence of the second number of oligonucleotide chains.

### EXAMPLES

Various aspects of the present invention can be better understood by reference to the following Examples which are offered by way of illustration. The inventive concepts described in the application are not limited to the Examples given herein.

### Example 1

### Polyaniline Electrochemical Polymerization

### Materials

Sulphuric acid (H₂SO₄, CAS 7664-93-9, 95-98%, ACS reagent) and aniline (C₆H₅NH₂, CAS 62-53-3, >99%, ACS reagent) was mixed with deionized water. A potentiostat (Autolab PGSTAT200, Metrohm Inc.), was used to control the voltages applied to the electrodes. The electrodes were comprised of stainless steel having a gold (Au) coating and low temperature cofired ceramics coated having a gold coating.

### Procedure

Aniline was distilled prior to the synthesis to remove unwanted byproducts. The distilled aniline was added to a diluted solution of H₂SO₄ such as to obtain a final 0.1 M aniline in 0.5 M H₂SO₄ solution (polymerization solution). The solutions were added to a standardized cell made of PTFE, where two electrodes were placed. The electrodes were connected to the potentiostat using alligator clips. The polymerization was carried out using cyclic voltammetry, with the parameters set as shown in Table 1.

TABLE 1
Parameters for the electrochemical polymerization of aniline

| Parameter | Value | |
|---|---|---|
| Start potential | 1.5 | V |
| Upper vertex potential | 1.8 | V |
| Lower vertex potential | 1.2 | V |
| Stop potential | 1.5 | V |
| Number of stop crossings | 14 or 15 | |
| Step potential | 2.5 | mV |
| Scan rate | 50 | mV/s |

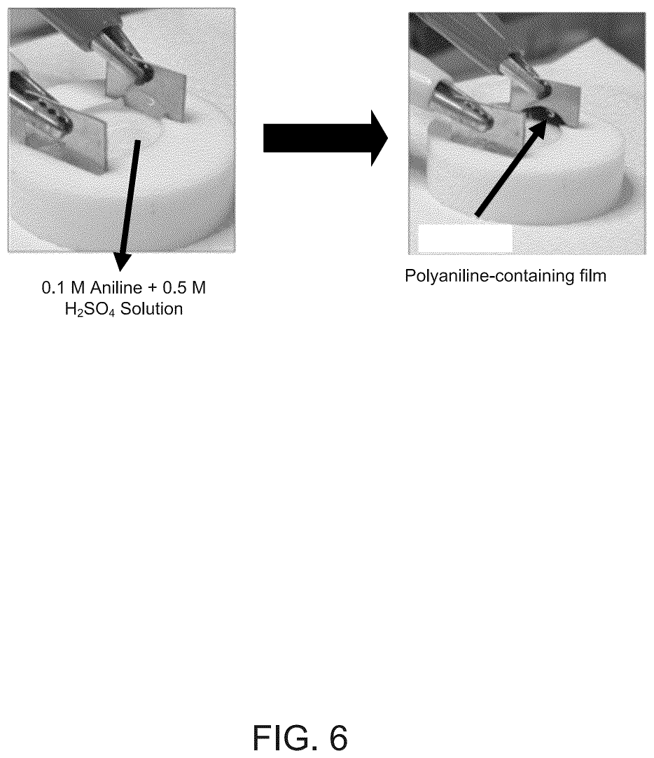

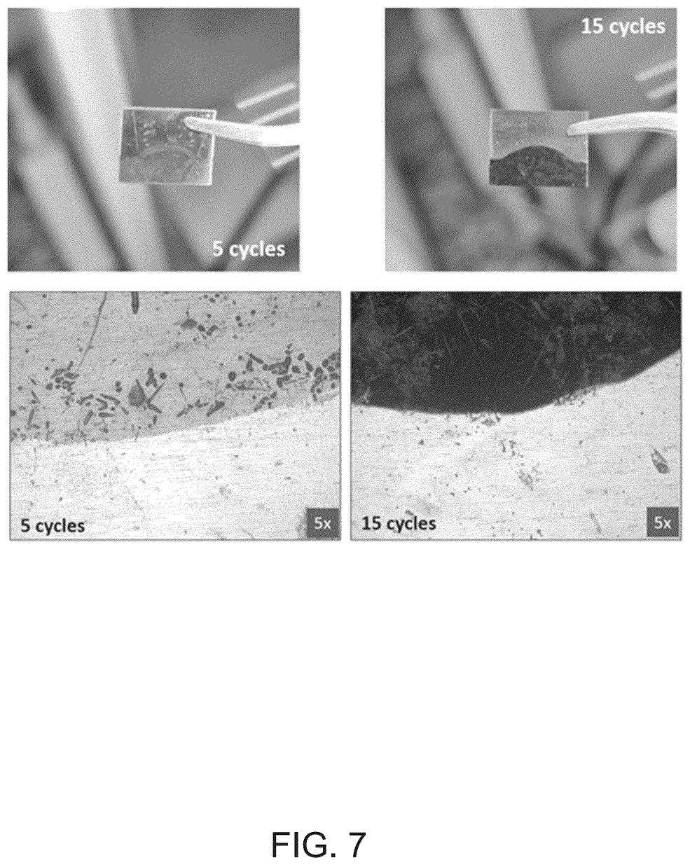

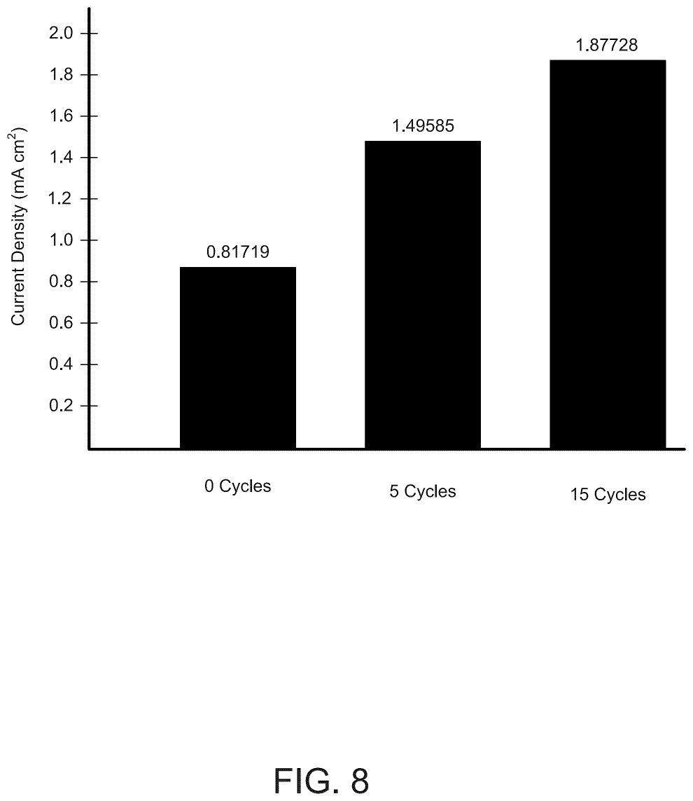

FIG. 6 shows the setup of the electrodes coupled to a potentiometer and contacting a coating solution having 0.1 M aniline monomer content and 0.5 M H₂SO₄ and the creation of the polyaniline-containing film after performing 15 cycles according to the conditions of Table 1. FIG. 7 shows images of electrodes coated with a polyaniline-containing film after applying 5 voltage cycles and after applying 15 voltage cycles at both 1× magnification and 5× magnification. FIG. 8 shows a graphic that indicates the increase in current density as the number of voltage cycles applied to the electrode went from 0 cycles to 5 cycles to 15 cycles.

### Example 2

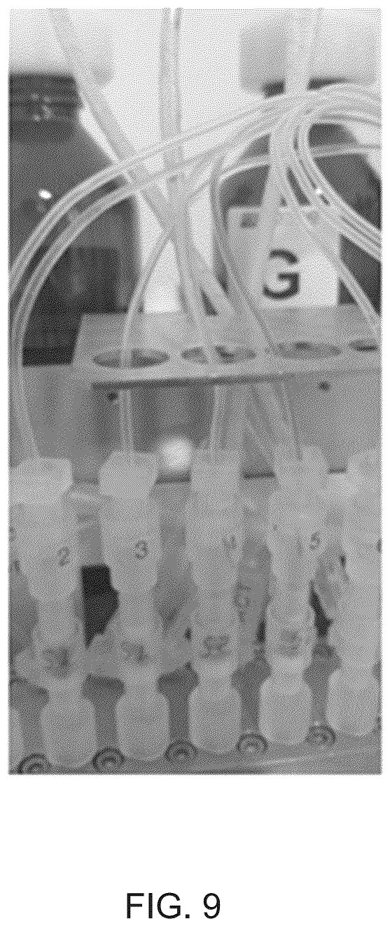

Oligonucleotides are synthesized using stainless steel electrodes that included a polyaniline coating formed according to Example 1. The stainless steel electrodes were placed into synthesis columns and a synthetic oligonucleotide having the following sequence from 5′ to 3′, were synthesized using the apparatus shown in FIG. 9:
CACGACGTTGTAAAACGACTCGTGGCAGATCAGTCCATAGCCGTCCAGACAAGAAC AGTACGGCCAAGAACATATCGTCCCAGATCCGACCATATCCTCTCTGATACGCCATA TAACACAGAGGGATGGGTCATAGCTGTTTCCTG.

Amplification and quantification operations were performed. Table 2 below shows results from a quantification analysis of the samples after synthesis and amplification operations are performed indicating a concentration of a synthetic oligonucleotide in nanograms per microliter (ng/μL) produced using polyaniline-coated electrodes for a number of samples.

TABLE 2
Quantification results for oligonucleotide concentration

| Sample | | | | | |
|---|---|---|---|---|---|
| Name | ng/uL | A260/A280 | A260/A230 | A260 | A280 |
| Sample 5 | 4851.6 | 1.66 | 1.04 | 147.02 | 88.33 |
| Sample 6 | 230.5 | 1.74 | 0.84 | 6.99 | 4.00 |
| Sample 7 | 83.9 | 1.78 | 0.65 | 2.54 | 1.43 |
| Sample 8 | 3642.7 | 1.60 | 1.02 | 110.38 | 69.01 |
| Sample 9 | 3461.5 | 1.66 | 1.11 | 104.89 | 63.35 |
| Sample 10 | 7051.7 | 1.63 | 1.02 | 213.69 | 131.34 |

### Example Aspects of the Disclosure

The following exemplary aspects are provided, the numbering of which is not to be construed as designating levels of importance:

Aspect 1. An oligonucleotide synthesis apparatus comprising: a reaction vessel; and an array of electrodes disposed in the reaction vessel, individual electrodes of the array of electrodes having a coating that includes a polyaniline-containing material.

Aspect 2. The oligonucleotide synthesis apparatus of aspect 1, wherein individual electrodes of the array of electrodes include a conductive material.

Aspect 3. The oligonucleotide synthesis apparatus of aspect 2, wherein individual electrodes of the array of electrodes includes a metallic material.

Aspect 4. The oligonucleotide synthesis apparatus of aspect 3, wherein the metallic material includes gold.

Aspect 5. The oligonucleotide synthesis apparatus of aspect 3, wherein the metallic material includes stainless steel.

Aspect 6. The oligonucleotide synthesis apparatus of aspect 2, wherein individual electrodes of the array of electrodes include an indium-tin-oxide material.

Aspect 7. The oligonucleotide synthesis apparatus of aspect 2, wherein individual electrodes of the array of electrodes include a fluorine-doped tin oxide material.

Aspect 8. The oligonucleotide synthesis apparatus of any one of aspects 1-7, wherein the polyaniline-containing material includes an emeraldine salt form of polyaniline.

Aspect 9. The oligonucleotide synthesis apparatus of any one of aspects 1-8, wherein one or more intermediate oligonucleotide chains are coupled to individual electrodes of the array of electrodes, individual intermediate oligonucleotide chains of the one or more intermediate oligonucleotide chains having a protecting group on a terminal nucleotide.

Aspect 10. The oligonucleotide synthesis apparatus of aspect 9, comprising a voltage source coupled to the array of electrodes, wherein the voltage source applies a voltage to one or more electrodes of the array of electrodes.

Aspect 11. The oligonucleotide synthesis apparatus of aspect 10, wherein: a deblocking solution is disposed in the reaction vessel; and applying the voltage to the one or more electrodes causes removal of the protecting group of the individual oligonucleotide chains attached to the one or more electrodes.

Aspect 12. The oligonucleotide synthesis apparatus of any one of claims 1-11, wherein the coating has an electrical conductivity at 20° C. of no greater than 6×10⁶ siemens per meter (S/m).

Aspect 13. A method comprising: providing a substrate with one or more electrodes disposed on the substrate; contacting the one or more electrodes with a coating solution, the coating solution having an acid content and a monomer content, the monomer content including a number of aniline monomer units; and applying a voltage to the one or more electrodes to form a coating on the one or more electrodes, the coating including a polyaniline-containing material.

Aspect 14. The method of aspect 10, wherein the acid content is comprised of from about 0.05 M to 0.2 M sulfuric acid (H₂SO₄).

Aspect 15. The method of aspect 10, wherein acid content is comprised of from about 0.05 M to 0.2 M hydrochloric acid (HCl).

Aspect 16. The method of any one of aspects 13-15, wherein the monomer content comprises from about 0.05 M to about 1 M aniline monomer units.

Aspect 17. The method of any one of aspects 13-16, wherein the coating solution is an aqueous solution having at least 50% by weight H₂O.

Aspect 18 The method of any one of aspects 13-17, wherein the coating solution comprises a polar aprotic solvent.

Aspect 19. The method of aspect 18, wherein the polar aprotic solvent comprises acetonitrile.

Aspect 20. The method of any one of aspects 13-19, wherein the coating solution comprises an electrolyte.

Aspect 21. The method of aspect 20, wherein the electrolyte comprises sodium perchlorate.

Aspect 22. The method of any one of aspects 13-21, wherein applying a voltage to the one or more electrodes to form the coating on the one or more electrodes includes applying one or more voltage cycles to the one or more electrodes.

Aspect 23. The method of aspect 22, wherein: a voltage cycle of the one or more voltage cycles includes increasing a voltage applied to the one or more electrodes from a lower threshold voltage to an upper threshold voltage at a voltage rate and decreasing the voltage applied to the one or more electrodes from the upper threshold voltage to the lower threshold voltage; and the voltage rate is from about 0.02 millivolts per second (mV/s) to about 0.08 mV/s.

Aspect 24. The method of aspect 23, wherein the lower threshold voltage is from about −0.6 V to about 0 V and the upper threshold voltage is from about 0.8 V to about 1.6 V.

Aspect 25. The method of aspect 23 or 24, wherein from about 8 voltage cycles to about 20 voltage cycles are applied to the one or more electrodes to form the coating.

Aspect 26. A method comprising: applying a voltage to an electrode disposed on a surface of a reaction vessel of an oligonucleotide synthesizer apparatus, the electrode having a coating that includes a polyaniline-containing material and a plurality of intermediate oligonucleotide chains being coupled to the electrode; and responsive to the voltage being applied to the electrode, causing a protecting group coupled to a terminal nucleotide of an intermediate oligonucleotide chain of the plurality of intermediate oligonucleotide chains to separate from the terminal nucleotide and form an unprotected intermediate oligonucleotide chain.

Aspect 27. The method of aspect 26, comprising: responsive to the voltage being applied to the electrode, causing an oxidation state of the coating to change from a reduced state to an oxidized state.

Aspect 28 The method of aspect 26 or 27, comprising: adding a synthesis solution to the reaction vessel, the synthesis solution including a nucleoside phosphoramidite, wherein the nucleoside phosphoramidite couples to the unprotected intermediate oligonucleotide chain.

Aspect 29. The method of any one of aspects 26-28, wherein a deblocking solution is disposed in the reaction vessel during the voltage being applied to the electrode, the deblocking solution including at least one of an amount of benzoquinone or an amount of hydroquinone.

Aspect 30. The method of aspect 29, wherein the deblocking solution includes at least one of an amount of tetrabutylammonium hexafluorophosphate, an amount of acetonitrile, or an amount of N,N-diisopropylethylamine.

Aspect 31. The method of aspect 28, wherein the nucleoside phosphoramidite corresponds to a nucleotide to add to the intermediate oligonucleotide chain in relation to an oligonucleotide sequence that encodes an amount of digital information.

Aspect 32. The method of aspect 28, wherein: an array of electrodes is disposed on one or more surfaces of the reaction vessel, the array of electrodes including the electrode; applying the voltage to the electrode includes applying the voltage to a first group of electrodes of the array of electrodes; and the first group of electrodes being coupled to a first number of intermediate oligonucleotide chains.

Aspect 33. The method of aspect 32, wherein: a first nucleotide is to be added to the first number of intermediate oligonucleotide chains to encode a first set of digital information using a sequence of the first number of intermediate oligonucleotide chains; and the nucleoside phosphoramidite corresponds to the first nucleotide.

Aspect 34. The method of aspect 33, wherein, subsequent to adding the first nucleotide to the first number of intermediate oligonucleotide chains, an additional voltage is applied to a second group of electrodes, at least one electrode of the second group of electrodes being different from at least one electrode of the first group of electrodes to add a second nucleotide to a second number of intermediate oligonucleotide chains attached to the second group of electrodes.

Aspect 35. The method of aspect 34, wherein the second nucleotide is added to the second number of intermediate oligonucleotide chains to encode a second set of digital information using an additional sequence of the second number of oligonucleotide chains.

What is claimed is:

1. An oligonucleotide synthesis apparatus comprising:
a reaction vessel; and
an array of electrodes disposed in the reaction vessel, individual electrodes of the array of electrodes having a coating that includes a polyaniline-containing material.

2. The oligonucleotide synthesis apparatus of claim 1, wherein individual electrodes of the array of electrodes include a conductive material.

3. The oligonucleotide synthesis apparatus of claim 1, wherein the polyaniline-containing material includes an emeraldine salt form of polyaniline.

4. The oligonucleotide synthesis apparatus of claim 1, wherein one or more intermediate oligonucleotide chains are coupled to individual electrodes of the array of electrodes, individual intermediate oligonucleotide chains of the one or more intermediate oligonucleotide chains having a protecting group on a terminal nucleotide.

5. The oligonucleotide synthesis apparatus of claim 4, comprising a voltage source coupled to the array of electrodes, wherein the voltage source applies a voltage to one or more electrodes of the array of electrodes;
wherein:
a deblocking solution is disposed in the reaction vessel; and
applying the voltage to the one or more electrodes causes removal of the protecting group of the individual oligonucleotide chains attached to the one or more electrodes.

6. The oligonucleotide synthesis apparatus of claim 1, wherein the coating has an electrical conductivity at 20° C. of no greater than 6×10⁶ siemens per meter (S/m).

7. A method comprising:
providing a substrate with one or more electrodes disposed on the substrate;
contacting the one or more electrodes with a coating solution, the coating solution having an acid content and a monomer content, the monomer content including a number of aniline monomer units; and
applying a voltage to the one or more electrodes to form a coating on the one or more electrodes, the coating including a polyaniline-containing material.

8. The method of claim 7, wherein the acid content is comprised of from about 0.05 M to 0.2 M sulfuric acid (H₂SO₄).

9. The method of claim 7, wherein acid content is comprised of from about 0.05 M to 0.2 M hydrochloric acid (HCl).

10. The method of claim 7, wherein the monomer content comprises from about 0.05 M to about 1 M aniline monomer units.

11. The method of claim 7, wherein the coating solution is an aqueous solution having at least 50% by weight H₂O.

12. The method of claim 7, wherein the coating solution comprises a polar aprotic solvent comprising acetonitrile.

13. The method of claim 7, wherein the coating solution comprises an electrolyte comprising sodium perchlorate.

14. The method of claim 7, wherein:
a voltage cycle of the one or more voltage cycles includes increasing a voltage applied to the one or more electrodes from a lower threshold voltage to an upper threshold voltage at a voltage rate and decreasing the voltage applied to the one or more electrodes from the upper threshold voltage to the lower threshold voltage; and
the voltage rate is from about 0.02 millivolts per second (mV/s) to about 0.08 mV/s.

15. The method of claim 14, wherein:
the lower threshold voltage is from about −0.6 V to about 0 V and the upper threshold voltage is from about 0.8 V to about 1.6 V; and
from about 8 voltage cycles to about 20 voltage cycles are applied to the one or more electrodes to form the coating.

16. A method comprising:
applying a voltage to an electrode disposed on a surface of a reaction vessel of an oligonucleotide synthesizer apparatus, the electrode having a coating that includes a polyaniline-containing material and a plurality of intermediate oligonucleotide chains being coupled to the electrode; and
responsive to the voltage being applied to the electrode, causing a protecting group coupled to a terminal nucleotide of an intermediate oligonucleotide chain of the plurality of intermediate oligonucleotide chains to separate from the terminal nucleotide and form an unprotected intermediate oligonucleotide chain.

17. The method of claim 16, comprising:
responsive to the voltage being applied to the electrode, causing an oxidation state of the coating to change from a reduced state to an oxidized state; and
adding a synthesis solution to the reaction vessel, the synthesis solution including a nucleoside phosphoramidite, wherein the nucleoside phosphoramidite couples to the unprotected intermediate oligonucleotide chain;
wherein a deblocking solution is disposed in the reaction vessel during the voltage being applied to the electrode, the deblocking solution including at least one of an amount of benzoquinone or an amount of hydroquinone.

18. The method of claim 17, wherein:
the deblocking solution includes at least one of an amount of tetrabutylammonium hexafluorophosphate, an amount of acetonitrile, or an amount of N,N-diisopropylethylamine; and
the nucleoside phosphoramidite corresponds to a nucleotide to add to the intermediate oligonucleotide chain in relation to an oligonucleotide sequence that encodes an amount of digital information.

19. The method of claim 17, wherein:
an array of electrodes is disposed on one or more surfaces of the reaction vessel, the array of electrodes including the electrode;
applying the voltage to the electrode includes applying the voltage to a first group of electrodes of the array of electrodes;
the first group of electrodes being coupled to a first number of intermediate oligonucleotide chains;
a first nucleotide is to be added to the first number of intermediate oligonucleotide chains to encode a first set of digital information using a sequence of the first number of intermediate oligonucleotide chains; and
the nucleoside phosphoramidite corresponds to the first nucleotide.

20. The method of claim 19, wherein:
subsequent to adding the first nucleotide to the first number of intermediate oligonucleotide chains, an additional voltage is applied to a second group of electrodes, at least one electrode of the second group of electrodes being different from at least one electrode of the first group of electrodes to add a second nucleotide to a second number of intermediate oligonucleotide chains attached to the second group of electrodes; and
the second nucleotide is added to the second number of intermediate oligonucleotide chains to encode a second set of digital information using an additional sequence of the second number of oligonucleotide chains.
